(19) United States
(12) Patent Application Publication
(10) Pub. No.: US 2010/0266691 A1
(43) Pub. Date: Oct. 21, 2010

(54) Agents and Methods to Stimulate Bone Healing

(75) Inventor: Norman J. Karin, Richland, WA (US)

Correspondence Address: WELLS ST. JOHN P.S., 601 WEST FIRST AVENUE, SUITE 1300, SPOKANE, WA, 99201-3828, US

(73) Assignee: BATTELLE MEMORIAL INSTITUTE, Richland, WA (US)

(21) Appl. No.: 12/550,064

(22) Filed: Aug. 28, 2009

Related U.S. Application Data
(60) Provisional application No. 61/169,520, filed on Apr. 15, 2009.

(51) Int. Cl. A61K 9/00; A61K 31/66; A61K 38/16; A61P 19/00

(52) U.S. Cl. 424/487; 424/484; 514/120; 514/12

(57) ABSTRACT

An agent for stimulating bone healing including isolated lysophosphatidic acid (LPA) and a hydrogel. A method of enhancing bone healing including identifying a damaged area of a bone and administering an agent comprising lysophosphatidic acid (LPA) is administered to the damaged area of the bone. A method of increasing bone regrowth. A subject is identified having a bone injury and lysophosphatidic acid (LPA) is administered to the subject.

### RELATED PATENT DATA

This patent claims benefit of priority under 35 U.S.C. §119 to U.S. Provisional Patent Ser. No. 61/169,520, which was filed Apr. 15, 2009.

### GOVERNMENT RIGHTS STATEMENT

This invention was made with Government support under contract DE-AC0676RLO1830 awarded by the U.S. Department of Energy. The Government has certain rights in the invention.

### TECHNICAL FIELD

The invention pertains to agents administered to stimulate bone healing, methods of enhancing bone healing and methods of increasing bone regrowth.

### BACKGROUND OF THE INVENTION

Millions of human bone fractures are sustained each year, many of which exhibit impaired healing that may require surgical intervention. Millions more fractures are seen by those in the veterinary medicine field. Skeletal defects are also introduced during tumor resections and the reconstruction of congenital defects. Dental implants also heal by bone regrowth. There is therefore a need for materials that can safely and effectively stimulate bone healing. Ease of handling and expense of materials are also factors to be considered. The demand for low-cost bone regrowth treatments also includes veterinary medicine where third party payment of treatment is rare.

Bone implant treatment materials available as injectable drug delivery systems could advantageously offer the ability to treat closed fractures without invasive and expensive surgery.

Current therapeutic agents available for stimulating bone regrowth are cost prohibitive to manufacture at this time. Available protein factors used to stimulate regrowth are often fragile and are susceptible to loss of function. It is desirable to develop alternative bone regrowth stimulating therapeutic agents and methods for stimulating bone regrowth.

### SUMMARY OF THE INVENTION

In one aspect, the invention encompasses an agent for stimulating bone healing including isolated lysophosphatidic acid (LPA) and a hydrogel.

In one aspect, the invention encompasses a method of enhancing bone healing. A damaged area of a bone is identified and an agent comprising lysophosphatidic acid (LPA) is administered to the damaged area of the bone.

In one aspect, the invention encompasses a method of increasing bone regrowth. A subject is identified having a bone injury and lysophosphatidic acid (LPA) is administered to the subject.

### BRIEF DESCRIPTION OF THE DRAWINGS

Preferred embodiments of the invention are described below with reference to the following accompanying drawings.

FIG. 1 shows the stimulation of pre-osteoblast chemotaxis by lysophosphatidic acid. *p=0.0029 vs. control; **p=0.0065 and ***p=0.0114 vs. LPA alone.

FIG. 2 shows gene ontology analysis of changes in pre-osteoblast gene expression after treatment with lysophosphatidic acid. The number of genes in each functional category exhibiting statistically-significant LPA induced up- or down-regulation is indicated to the right of the bars.

FIG. 3 shows induction of osteocyte membrane outgrowth by lysophosphatidic acid. Data are normalized to untreated control cells.

FIG. 4 shows MicroCT images of rat femurs six weeks after implantation with PLGA scaffolds+/−LPA or BMP-2. Defect regions (arrows) are enlarged on the right; dotted lines depict the original defect boarders.

FIG. 5 shows MicroCT images of rat femurs four weeks after implantation with PLGA scaffolds plus nothing (control, panel A) LPA (Panel B), BMP-2 (Panel C) or both LPA and BMP2 (Panel D).

FIG. 6 shows MicroCT images of rat femurs four weeks after implantation with PLGA scaffolds plus nothing (control, panel A) LPA (Panel B), BMP-2 (Panel C) or both LPA and BMP2 (Panel D).

### DETAILED DESCRIPTION OF THE PREFERRED EMBODIMENTS

This disclosure of the invention is submitted in furtherance of the constitutional purposes of the U.S. Patent Laws “to promote the progress of science and useful arts” (Article 1, Section 8).

In general, the invention pertains to therapeutic agents for stimulating bone healing and/or regrowth and methods of stimulating bone regrowth and/or healing of damaged bone. For purposes of the present description, bone damage can refer to damage due to trauma, injury, dental procedures, tumor resections, bone reconstruction and the like. The therapeutic agents of the invention include lysophosphatidic acid (LPA).

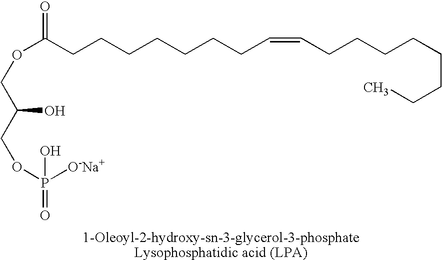

LPA is a lipid growth factor which is relatively inexpensive to produce as compared to protein growth factors that have been shown to affect bone growth. LPA is additionally relatively stable as compared to protein growth factors.

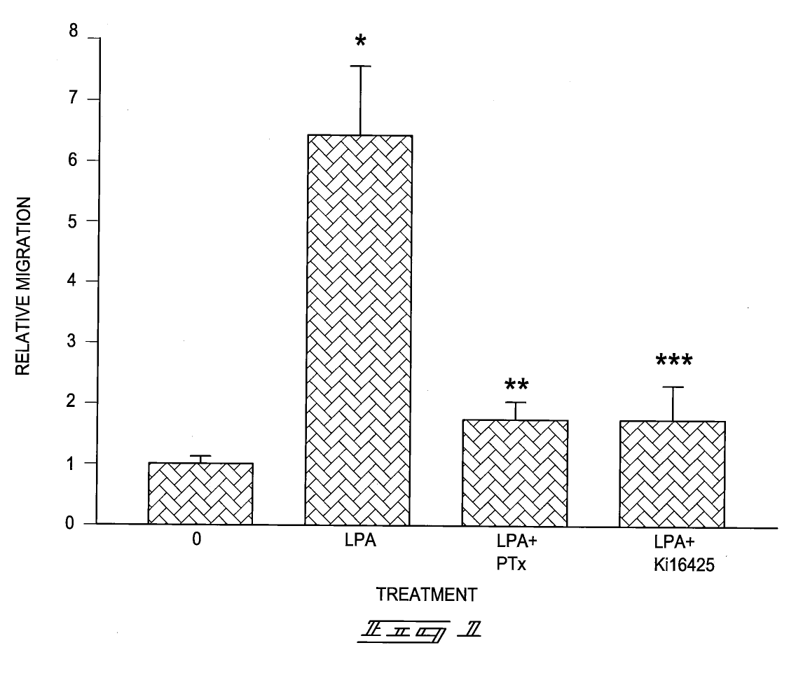

LPA has been shown to have a stimulating effect on the activity of bone-forming osteoblasts in vitro. LPA is also a potent enhancer of angiogenesis and neovascularization is essential for proper bone healing. LPA additionally exhibits chemotactic effects on pre-osteoblast cells (see FIG. 1).

Mouse MC3T3-E1 pre-osteoblastic cells were seeded into the upper wells of opaque trans-well chambers that had 8.0-micron pores. LPA was added to the growth medium in the lower chamber. The number of cells that migrated through the pores and attached to the underside of the microporous membrane served as a measure of chemotaxis (chemically directed cell migration). Cells were fluorescently labeled with calcein to allow visualization on the undersides of the opaque chambers. Utilizing this assay it was determined that LPA elicited a 6.5 fold increase in pre-osteoblast when compared to cells under control conditions (growth medium in the absence of LPA).

The decreased chemotactic effect in the presence of inhibitors pertussis toxin (PTx) and LPA receptor antagonist (Ki16425) indicates that the migratory response of the pre-osteoblasts is mediated by G protein coupled receptors.

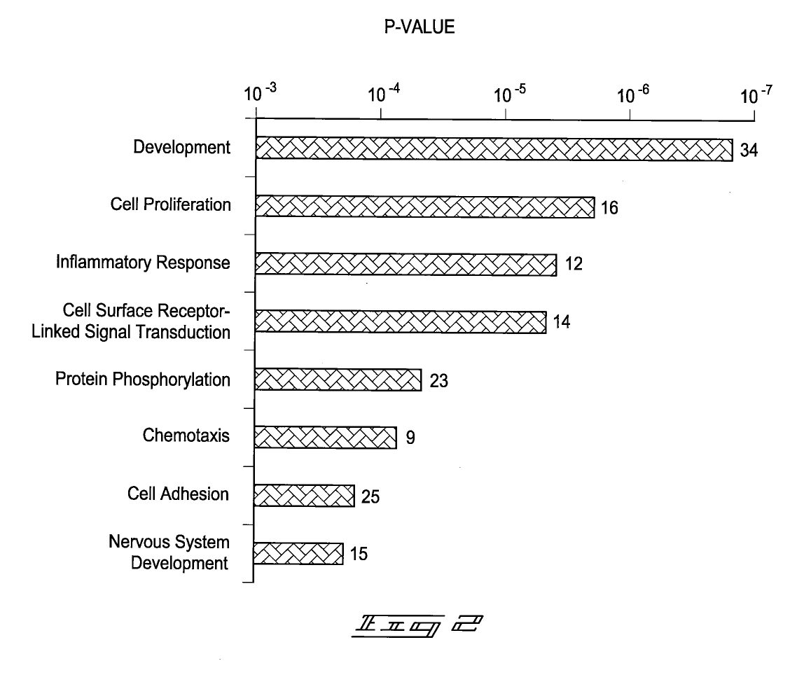

LPA also modulates the expression of a large set of pre-osteoblast genes linked to development (FIG. 2). MC3T3-E1 cells were grown in the presence or absence of a physiological level of LPA (1.0 μM). After growth of the cells, total RNA was isolated for use in global gene expression profiling. Gene expression was measured utilizing Affymetrix mouse whole genome microarrays. Data were analyzed using a suite of bioinformatics software. 513 genes were found to be regulated in LPA-treated cells. As shown, genes involved in cell development are the most statistically significant group affected by LPA. This study also revealed that LPA may have a significant role in osteoblast maturation and suppression of inflammation.

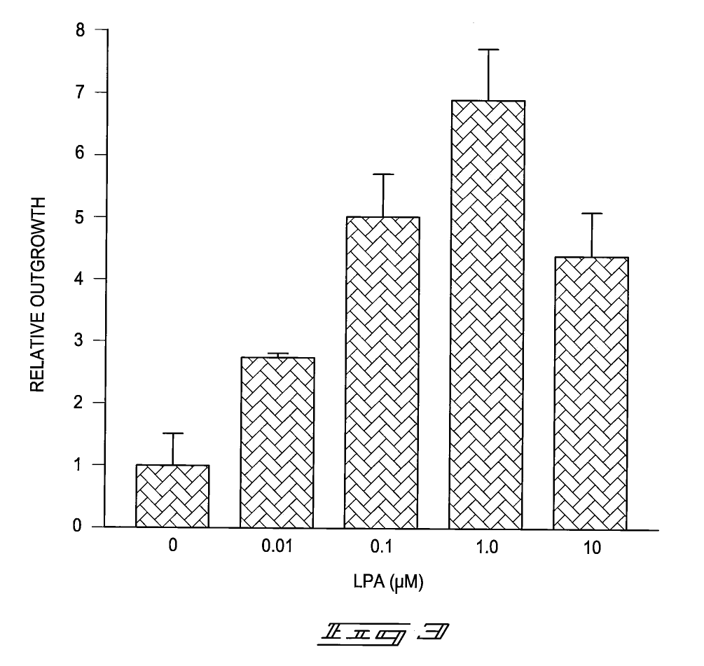

Among the effects of LPA on pre-osteoblasts is the induction of membrane extensions that resemble osteocyte dendrites. An assay was developed to study the ability of LPA to stimulate the outgrowth of dendritic processes from osteocytes in vitro, FIG. 3. Mouse MLO-Y4 osteocyte-like cells were seeded in the upper wells of opaque trans-well chambers having 1.0-micron pores which prevent the passage of whole cells but allow penetration by membrane extensions. Cells were fluorescently labeled with calcein to allow visualization of dendrites that were extended onto the undersides of the opaque chambers. It was determined that the presence of LPA in the lower chamber lead to a 7-fold increase in osteocyte dendrite outgrowth.

LPA significantly stimulated dendritogenesis at each concentration tested. It is postulated that LPA enhances dendrite outgrowth in vivo, which can re-establish the osteocyte mechanosensory network during the regeneration of damaged bone.

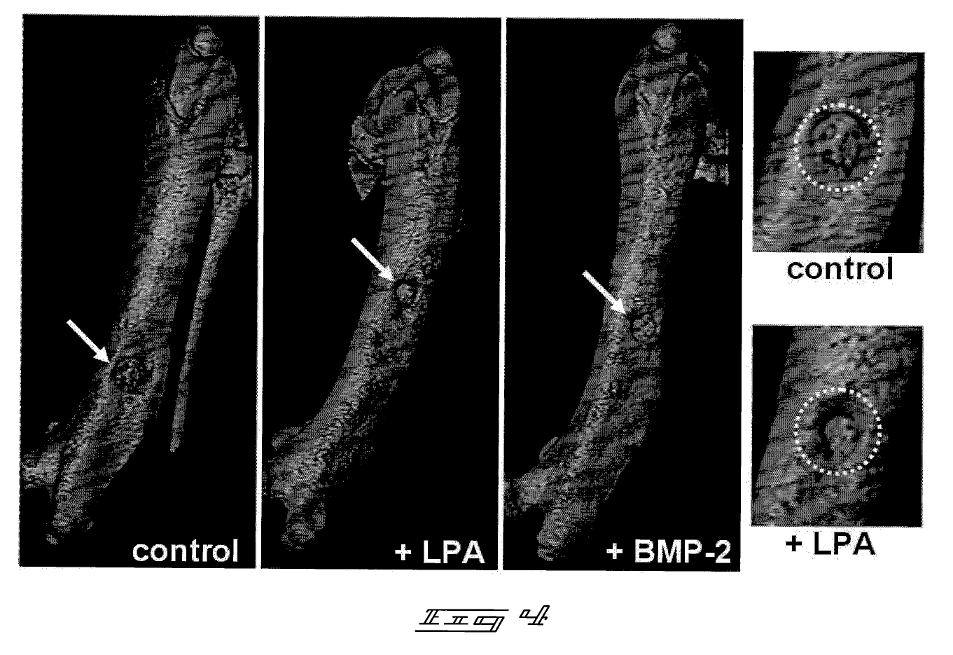

The inventor has shown, using rat models, that LPA enhances skeletal regeneration in vivo. The bone growth stimulatory effects of LPA were compared to those of bone morphogenetic protein 2 (BMP-2), which is an FDA approved osteoinductive additive to bone implants. Referring to FIG. 4, drill-hole defects (3-mm diameter) were introduced into the cortex of rat femurs. Implants were fabricated utilizing poly(DL-lactic-co-glycolic acid) copolymers) (PLGA). The femurs were then implanted with either LPA or a therapeutic amount (2 μg) of BMP-2 and were compared to a non-treated control. Bone healing was measured as the rate of closure of the defect as visualized in living animals by micro-computed tomography (microTC). Implants were designed as follows: PLGA alone; PLGA+12.5 μg LPA; PLGA+2.0 μg rhBMP2. FIG. 4 shows the resulting healing after six weeks post-treatment. From the results it appears that the stimulatory properties of LPA exceed those of BMP-2.

LPA is produced by platelets in blood clots during wound hemostasis. LPA is produced at the site of hematoma immediately following a bone injury. However, during surgery to repair a damaged bone, the surgeon typically removes blood clots thereby removing the natural source of LPA. LPA is also abundant in serum and is likely to contribute toward the biological activity of “platelet-rich plasma” which is a potent stimulator of fracture healing when added to bone implants.

In accordance with the invention, LPA can be administered to a subject by a variety of forms and utilizing any of a number of routes. LPA can be in a solid, liquid or aerosol form for administration, or can be applied as a coating. Administration routes include but are not limited to orally, topically, enterally, parenterally, nasally, rectally, etc. It can be preferable to administer the LPA directly in the area or at the site of bone injury. Accordingly, a therapeutic agent containing LPA can be administered directly to the damaged site during surgery or can be injected to the damaged site in an absence of surgery or following surgery. Formulations for administration will depend upon the route of administration.

For administration at the site or area of bone damage, LPA can be administered by injection into closed fractures, injection into craniofacial bone to accelerate integration of dental implants, or can be injected or otherwise applied to open fractures. It can be preferable that the LPA be released immediately upon administration or be controlled release over a period of from about 48 hrs, 72 hrs, 96 hrs, etc., up to greater than a period of seven days. One preferred method for controlled release is to utilize biodegradable matrices containing LPA. One preferred matrix is PLGA (described above). Another preferred matrix can be a hydrogel which can be implanted at the site of bone damage.

The invention is not limited to use of a particular hydrogel. Hydrogels which can be utilized include those hydrogels described in U.S. Pat. No. 6,979,464, hereby incorporated by reference. The described hydrogels are therapeutic agent carriers that comprise copolymer of [meth-]acrylamide derivative and a hydrophilic co-monomer, where [meth-]acrylamide refers to methacrylamide, acrylamide, or combinations thereof. The hydrogels are thermally reversible, are in the gel form at physiological temperature and are liquid below physiological temperature. Accordingly, the hydrogel containing LPA can be administered at a sub-physiological temperature, such as about 4° C., and can gel upon reaching physiological temperature. The hydrogel can be formulated to release the therapeutic LPA over a desired controlled time range such as 48 hours, 72 hours, 96 hours, etc up to a time period of greater than seven days.

A presently preferred embodiment comprises a method of treatment of bone damage utilizing application of a composition comprising a hydrogel and a therapeutic amount of LPA wherein the therapeutic amount of LPA is from between about 10 μg and about 100 mg of LPA per unit dose. The therapeutic amount utilized can depend human vs. animal use and upon the size of the animal for veterinary use. In particular application, a preferred therapeutic amount can be from about 0.1 mg to about 10 mg per unit dose.

LPA can alternatively be incorporated into coatings of bone and dental graft material. Example graft coating materials include but are not limited to ceramics and biodegradable copolymers. Since LPA stimulates angiogenesis, the addition of LPA-containing coatings on bone grafts may increase the success of this procedure: incomplete vascularization of bone grafts is a leading cause of failure.

In accordance with another aspect, bone regrowth can be enhanced by administering LPA in combination with one or more additional factors which influence bone healing. LPA in combination with other growth factors can have a cumulative effect or a synergistic effect on bone regrowth. Factors with which LPA can be combined include but are not limited to bone morphogenetic protein (BMP)-2, BMP-4, BMP-7, vascular endothelial growth factor (VEGF), platelet derived growth factor (PDGF), insulin-like growth factor (IGF), fibroblast growth factor (FGF), transforming growth factor-beta 1 (TGF-β1), and Nell-1.

Bone morphogenetic proteins (BMPs) are widely studied as stimulators of bone regeneration. There are approximately 15 known members of the BMP family and many have been shown to have osteoinductive properties. BMP-2 and BMP-7 (OP-1) currently are approved for clinical use in humans. Therapeutic amounts of BMPs in humans are typically 3 mg through 12 mg per dose. BMPs stimulate the differentiation of bone cell progenitors and trigger the secretion of the angiogenic growth factor VEGF. Both of these phenomena contribute toward bone healing. Recent data suggest that BMP-2 is particularly important for the early stages of bone healing.

LPA can be administered in combination with one or more BMPs to combine the osteoinductive properties of BMPs with the effects (set forth above) of LPA to have cumulative or synergistic effects on bone healing.

Vascular endothelial growth factor (VEGF) stimulates bone formation by increasing the extent of revascularization of the damaged region. New blood vessels not only provide nutrients and remove cellular waste, but also create conduits by which blood-borne skeletal precursor cells can be delivered to the regenerating area. As indicated above, LPA has angiogenic properties that are comparable to VEGF. LPA and VEGF can be combined and co-administered and can have a cumulative effect or synergistic effect upon bone regeneration and healing.

Platelet derived growth factor (PDGF), like LPA, is generated by platelets during blood clot formation in areas of bone damage. PDGF fosters bone healing by stimulating the proliferation of bone progenitor cells. A combination of LPA and PDGF can be administered and can have cumulative or synergistic effects upon bone regeneration and healing.

Insulin like growth factors IGF-1 and IGF-2 stimulate bone formation primarily via their mitogenic effects on bone precursor cells. IGF-1, IGF-2, or both can be co-administered with LPA and can produce a cumulative or synergistic effect upon bone regrowth and healing.

Fibroblast growth factors FGF-1 and FGF-2 have both mitogenic effects and angiogenic action that enhance bone healing. One or both of FGF-1 and FGF-2 can be combined with LPA and can be co-administered to produce cumulative or synergistic effects on bone regrowth and bone healing.

Transforming growth factor beta 1 (TGF-β1) is structurally and functionally related to the BMP family of osteogenic proteins. Its potential use as a clinical treatment to enhance bone healing is complicated by an apparent need for continuous dosing. However, combination of TGF-β1 and LPA and co-administration may show a cumulative or synergistic effect upon bone regrowth and bone healing.

Nell-1 is a recent addition to the group of protein growth factors shown to regulate bone formation. Nell-1 stimulates the growth and differentiation of osteoblast precursors by an as of yet unknown mechanism. Nell-1 is secreted, suggesting that it acts by binding to cell surface receptors but may also function intracellularly by binding to protein kinase C. LPA can be combined and co-administered with Nell-1 and can have cumulative or synergistic effects upon bone regrowth and bone healing.

The combination therapeutic agents can be in any of the forms (coating, gels, etc.) discussed above. In particular instances, the combinatorial agents can be in a hydrogel. The amount of protein growth factor(s) utilized in combination with LPA can be a therapeutic amount of the individual protein growth factor (the amount shown to effect bone regrowth when utilized individually). In some instances it can be possible to decrease the amount of protein growth factor utilized in combination with LPA to below its individual therapeutic amount due to the cumulative or synergistic effect of LPA.

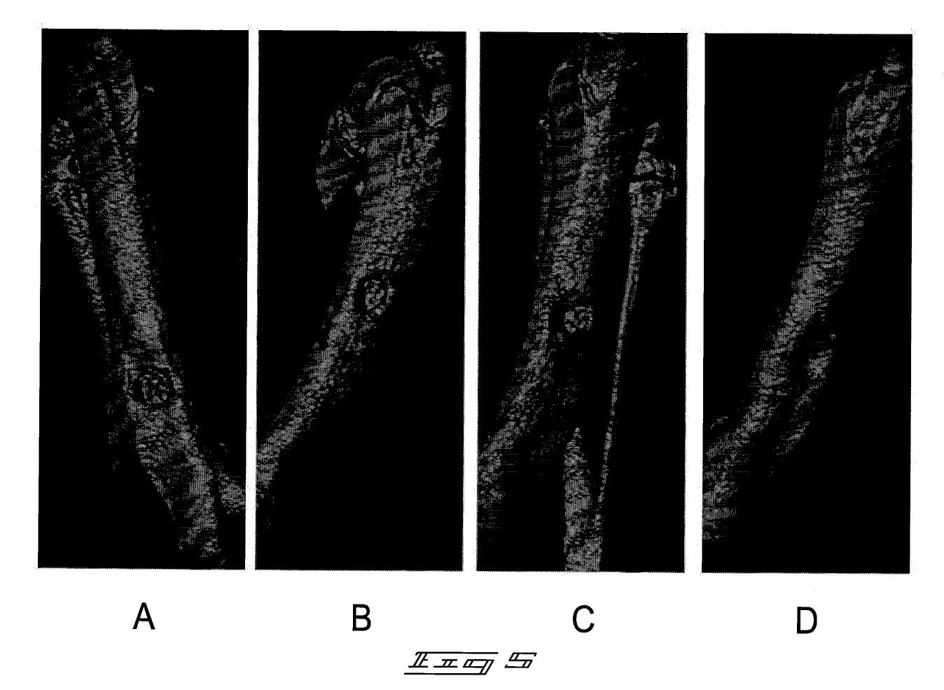

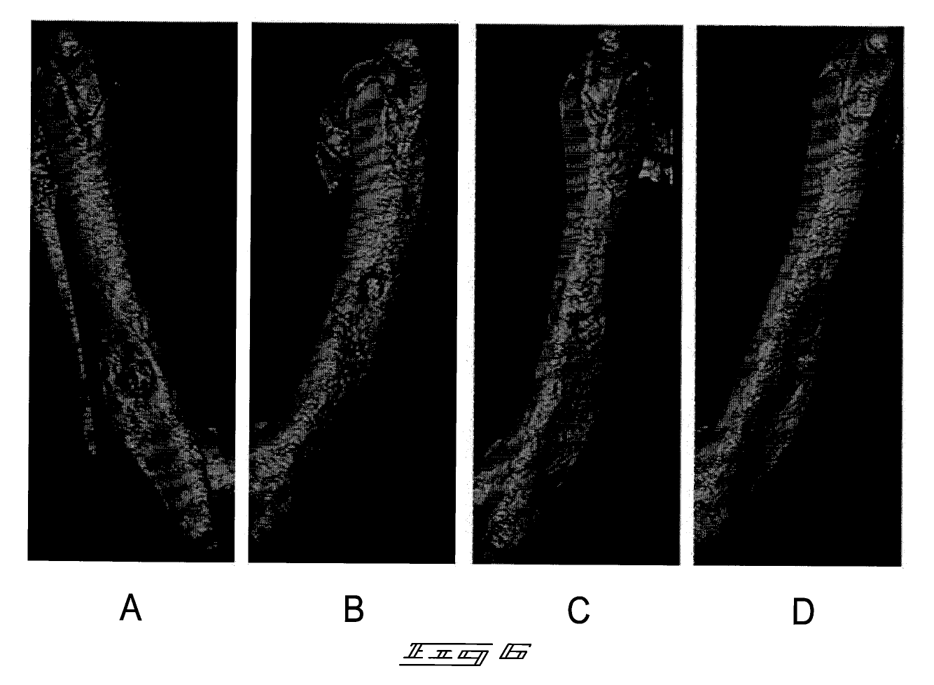

One preferred combination of the invention is LPA and BMP-2. Utilizing a rat model, the affects of LPA and LPA with BMP-2 were studied. Rat femurs were drilled to introduce a defect as set forth for FIG. 4. Implants were prepared as described above with the addition of an implant having PLGA+2.0 μg rhBMP2+12.5 μg LPA. Each corresponding drill drill-hole was implanted with: A) nothing (control); B) LPA; C) BMP-2 or D) LPA+BMP-2. Referring to FIG. 5, such shows the healing that occurred for each test sample after four weeks. Sample D (LPA+BMP2) shows a clear synergistic effect as compared to test cases B and C. FIG. 6 shows the results utilizing the same conditions after six weeks post treatment. It may be possible to improve these results by the addition of BMP-7 or another of the factors listed above to the combination of LPA and BMP-2.

Methods of the invention include identifying a subject having bone damage and administering a therapeutic amount of LPA. Identification of a subject can include conventional examination techniques as would be understood by one of ordinary skill in the art. Administration can comprise administration of any of the agents discussed above in any of the forms described.

LPA is a potent stimulator of bone regrowth and healing. It is relatively inexpensive and stable as compared to alternative osteoinductive agents. LPA can be utilized in conjunction with protein factors and can have a synergistic effect upon bone regeneration. Accordingly, LPA can be utilized individually or can be combined with small amounts of other agents to save costs of treatment while attaining improved bone regrowth.

In compliance with the statute, the invention has been described in language more or less specific as to structural and methodical features. It is to be understood, however, that the invention is not limited to the specific features shown and described, since the means herein disclosed comprise preferred forms of putting the invention into effect. The invention is, therefore, claimed in any of its forms or modifications within the proper scope of the appended claims appropriately interpreted in accordance with the doctrine of equivalents.

The invention claimed is:

1. An agent administered to stimulate bone healing comprising isolated lysophosphatidic acid (LPA) and a hydrogel.

2. The agent of claim 1 wherein the hydrogel is a liquid at 4° C. and a gel at physiological temperature.

3. An agent administered to stimulate bone healing comprising lysophosphatidic acid (LPA) and comprising one or more factors selected from the group consisting of bone morphogenetic protein (BMP)-2, BMP-4, BMP-7, vascular endothelial growth factor (VEGF), platelet derived growth factor (PDGF), insulin-like growth factor (IGF), fibroblast growth factor (FGF), transforming growth factor-beta 1 (TGF-β1), and Nell-1.

4. The agent of claim 3 wherein the one or more factors comprises BMP-2 and wherein the BMP-2 has a synergistic effect with LPA upon bone healing.

5. The agent of claim 3 further comprising a hydrogel.

6. A method of enhancing bone healing comprising:
identifying a damaged area of a bone; and
administering an agent comprising a therapeutic amount of lysophosphatidic acid (LPA) to the damaged area of the bone.

7. The method of claim 6 wherein the agent further comprises a hydrogel and wherein the administering comprises injecting the hydrogel to the area of bone damage.

8. The method of claim 7 the hydrogel is a liquid at 4° C. and a gel at physiological temperature.

9. The method of claim 6 further comprising one or more factors selected from the group consisting of bone morphogenetic protein (BMP)-2, BMP-4, BMP-7, vascular endothelial growth factor (VEGF), platelet derived growth factor (PDGF), insulin-like growth factor (IGF), fibroblast growth factor (FGF), transforming growth factor-beta 1 (TGF-β1), and Nell-1.

10. The method of claim 9 wherein the one or more factors comprises BMP-2 and wherein the BMP-2 has a synergistic effect with LPA upon bone healing.

11. A method of increasing bone regrowth, comprising:
identifying a subject having bone injury; and
administering a therapeutic amount of lysophosphatidic acid (LPA) to the subject.

12. The method of claim 11 wherein the administering is performed at the site of the bone injury.

13. The method of claim 11 wherein the administering is performed by injection.

14. The method of claim 11 wherein the LPA is carried by a carrier and wherein the LPA is released from the carrier at a controlled rate.

15. The method of claim 14 wherein the carrier comprises a hydrogel.

16. The method of claim 15 wherein the hydrogel comprises copolymer of [meth-]acrylamide derivative and a hydrophilic co-monomer.

17. The method of claim 15 wherein the hydrogel is a gel at physiological temperature and is a liquid at 4° C.

18. The method of claim 11 wherein the administering further includes administering one or more additional factors selected from the group consisting of bone morphogenetic protein (BMP)-2, BMP-4, BMP-7, vascular endothelial growth factor (VEGF), platelet derived growth factor (PDGF), insulin-like growth factor (IGF), fibroblast growth factor (FGF), transforming growth factor-beta 1 (TGF-β1), and Nell-1.

19. The method of claim 18 wherein the one or more additional factors have a synergistic effect with LPA on bone regrowth.

20. The method of claim 18 wherein the one or more additional factors include BMP-2.

21. The method of claim 18 wherein the one or more additional factors include BMP-2 and BMP-7.
